(19) United States
(12) Patent Application Publication
(10) Pub. No.: US 2009/0175789 A1
(43) Pub. Date: Jul. 9, 2009

(54) Antibody raised against the ldl receptor

(75) Inventors: Christian Behrens, Palaiseau (FR); Christine Gaucher, Sequeden (FR); Jean-Francois Prost, Versailles (FR); Jamila Najib, Santes (FR)

Correspondence Address: HARNESS, DICKEY & PIERCE, P.L.C., P.O. BOX 828, BLOOMFIELD HILLS, MI, 48303, US

(73) Assignee: LFB Biotechnologies, Les Ulis (FR)

(21) Appl. No.: 11/989,821

(22) PCT Filed: Jul. 25, 2006
(86) PCT No.: PCT/FR2006/001806

§ 371 Date: Jan. 31, 2008

(30) Foreign Application Priority Data
Aug. 3, 2005 (FR) 0508282

(51) Int. Cl. A61K 49/00; C07K 16/28; C12N 5/06; C07H 21/00; C12N 15/63; C07K 14/47; A61K 39/395; C12Q 1/02; G01N 33/53

(52) U.S. Cl. 424/9.1; 530/387.9; 530/387.3; 530/391.7; 435/331; 536/23.53; 435/320.1; 530/300; 424/139.1; 435/29; 435/7.1; 435/7.92

(57) ABSTRACT

The present invention relates to a monoclonal antibody raised against the human LDL (Low Density Lipoprotein) receptor, binding to the peptide corresponding to amino acids 195-222 (SEQ ID NO: 1) in the peptide sequence for the human LDL receptor; to the use thereof as a drug; to a pharmaceutical composition containing this antibody; and to the use thereof in immunohistochemical analyses of cancerous, healthy, or cirrhotic tissues, in Western-Blot or ELISA analyses, or in in vivo quantification tests.

### BACK GROUND OF THE INVENTION

The present invention relates to a monoclonal antibody raised against the human LDL (Low Density Lipoprotein) receptor, binding to the peptide corresponding to amino acids 195-222 (SEQ ID NO: 1) in the peptide sequence for the human LDL receptor; to the use thereof as a drug; to a pharmaceutical composition containing this antibody; and to the use thereof in immunohistochemical analyses of cancerous, healthy, or cirrhotic tissues, in Western-Blot or ELISA analyses, or in in vivo quantification tests.

Cholesterol is a lipid synthesized by the liver, the intestine, and the adrenal glands, but it is also provided by food. It is involved in the synthesis of sex hormones, corticosteroids such as natural cortisone, and bile constituents.

Insoluble in blood, gamma cholesterol is transported by lipoproteins, particularly LDLs (Low Density Lipoproteins).

The cholesterol then penetrates the cell, thanks to a cell surface protein that is able to recognise the LDLs: the LDL receptor (LDL-R). The LDL/LDL-R complex is then internalised by endocytosis, and the LDLs are digested by the lysosomes, releasing the cholesterol for use by the cell.

Cholesterolemia refers to the level of cholesterol in the blood. Hypocholesterolemia refers to a deficiency of cholesterol in the blood, whereas hypercholesterolemia refers to an excess of cholesterol in the blood.

It has been demonstrated that hypercholesterolemia can occur due to a defect in binding between the LDLs and the LDL-R, or due to defective internalisation of the LDLs, which can be caused by a familial structural alteration in the LDL-R among these patients (Beisiegel et al., 1981).

Furthermore, studies have shown that patients with certain cancers suffer from hypocholesterolemia. This hypocholesterolemia is the result of overuse of cholesterol by cancer cells. For their survival, the latter induce an increase in the expression level of the LDL receptor (LDL-R) within the tumoural organs (Henricksson et al., 1989).

There is therefore a correlation between the increase in the LDL-R expression level by the cells and certain cancers. These include, particularly, prostate, breast, liver, pancreatic, ovarian, colon, lung, and stomach cancers, as well as leukemias.

Moreover, it is currently known that the endocytosis of the hepatitis-C virus is mediated by LDL-Rs. Thus, LDL-R can serve as a viral receptor.

Thus, it appears that LDL-R is involved in numerous important cell life mechanisms, as well as in numerous disease conditions.

Therefore, the study of LDL-R remains a major challenge, not only to understand its tissular expression profile in the disease conditions in which it is involved, but also with regard to the development and study of new therapeutic tools for the treatment of such disease conditions.

Accordingly, to meet this need, the present Applicant has sought to develop a new tool that exhibits both sensitivity and specificity for LDL-R, thus making it particularly well suited to the study of the expression of LDL-R, especially in immunohistochemical analyses of healthy, tumoural, or cirrhotic tissues, in Western-Blot and in ELISA analyses, or in in vivo quantification tests, or even in the development of new therapeutic tools, especially for use in cancer treatment.

Thus, the invention relates to a monoclonal antibody raised against the human LDL (Low Density Lipoprotein) receptor.

### SUMMARY OF THE INVENTION

A first object of the invention relates to a monoclonal antibody raised against the human LDL (Low Density Lipoprotein) receptor, binding to the peptide corresponding to amino acids 195-222 (SEQ ID NO.: 1) in the peptide sequence for the human LDL receptor.

### DETAILED DESCRIPTION OF THE INVENTION

The human LDL receptor (LDL-R) is a transmembrane protein consisting of 839 amino acids and including three regions: the extracellular region (1-768), the transmembrane region (768-790), and the cytoplasmic region (790-839). The extracellular region is divided into two sub-regions: the LDL-binding sub-region (1-322) and the sub-region outside the LDL-binding region (322-768).

The antibody according to the invention was produced so as to bind specifically to the peptide corresponding to amino acids 195-222 (SEQ ID NO: 1) in the LDL-R peptide sequence. This peptide is located in the LDL-binding region. This peptide was selected because it displays good accessibility to the antibody according to the invention, because of its location in the LDL-binding region, and because of its three-dimensional conformation. It also has the characteristics of being immunogenic, due to its amino-acid composition.

Thus, this peptide was selected as the target of the antibodies according to the invention, with a view toward producing an antibody that is a good competitor of LDLs and that therefore exhibits good affinity for LDL-R. This peptide also displays 85% homology with the murine LDL-R, which allows the production of antibodies that cross-react in humans and mice, thus offering the possibility to implement both tests (particularly toxicity tests) in mice and use in humans.

Other advantages of selecting this particular peptide will become clear upon review of the remainder of the description.

For the purposes of the invention, the peptide to which the antibody binds may correspond to a peptide included in the peptide corresponding to amino acids 195-222 (SEQ ID NO: 1) of LDL-R. More specifically, the term “peptide” should be understood as referring to any molecule formed by the concatenation of at least 2 amino acids, preferably 5 to 35 amino acids, and possibly more than 35 amino acids.

For the purposes of the invention, the phrase “monoclonal antibody” or “monoclonal antibody composition” refers to a preparation of antibody molecules exhibiting an identical and unique specificity.

Furthermore, “antibody” means any whole antibody, as well as any polypeptide, peptide, or protein containing at least one immunoglobulin domain or fragment, as well as any antibody derivative.

An immunoglobulin molecule consists of 4 polypeptides: 2 identical heavy (H) chains, each of 50 kDa, and 2 identical light (L) chains, each of 25 kDa. The light chain consists of 2 domains: a variable domain V and a constant domain C, which are folded in space independently of each other. They are called “VL” and “CL”. The heavy chain also contains one V domain, referred to as “VH”, and 3 or 4 C domains, referred to as “CH₁” through “CH₄”. Each domain contains approximately 110 amino acids, and is structured in a comparable manner. The 2 heavy chains are linked by disulfide bonds, and each heavy chain is also linked to a light chain by a disulfide bond.

The region that determines the specificity of the antibody for the antigen is carried by the variable parts, while the constant parts can interact with the Fc receptors of effector cells or of molecules such as the complement, to mediate various functional properties.

Thus, the phrase “immunoglobulin domain” refers to any one of the following domains: VL, CL, VH, CH₁, CH₂, CH₃, and CH₄. The antibody according to the invention may advantageously contain one or more of these domains, with all of the combinations of the above-mentioned domains falling within the scope of the invention.

The phrase “immunoglobulin fragment” refers to one of the fragments selected from among the Fab, Fab′, F(ab′)2, and Fc fragments, an scFv, or a CDR (Complementarity-Determining Region).

The enzymatic digestion of immunoglobulins by papain generates 2 identical fragments, which are referred to as the “Fab fragment” (Fragment Antigen Binding fragment), and the Fc fragment (crystallisable fragment). The Fc fragment supports the effector functions of immunoglobulins.

Through pepsin digestion, an F(ab′)2 fragment is generated, in which the two Fab fragment are linked by two disulfide bonds, and the Fc fragment is cleaved into multiple peptides. The F(ab′)2 fragment consists of two Fab′ fragments linked by inter-chain disulfide bonds to form an F(ab′)2.

Regarding the variable regions of the heavy and light chains, it can be seen that the sequence variability is not equally distributed. In fact, the variable regions consist, on the one hand, of very sparingly variable regions known as “framework” (FR) regions, of which there are 4 (FR1 to FR4), and on the other hand, of regions of extreme variability: these are the so-called “hypervariable” regions or CDRs, of which there are 3 (CDR1 to CDR3).

An scFv (Single Chain Fragment Variable) is a fragment that consists solely of the VH and VL variable domains of a monoclonal antibody, and whose structure is stabilised by a flexible short peptide arm located between the two domains (Billiald et al., 1995). Such fragments can be produced by bacteria. These molecules retain the ability to recognise an antigen in a specific manner. Small in size (29 kDa), they exhibit relatively low immunogenicity and are better tolerated than whole antibodies.

Thus, because the antibody according to the invention may advantageously contain one or more of these fragments, all the combinations of the above-mentioned fragments fall within the scope of the invention.

According to one particular aspect of the invention, the antibody according to the invention contains at least one immunoglobulin domain and at least one immunoglobulin fragment, for example, an Fc fragment and one or more variable or hypervariable regions.

Last, the term “antibody derivative” refers to any antibody that may contain one or more mutations, substitutions, deletions, and/or additions of one or more amino-acid residues.

The antibody according to the invention, which has the particular feature of binding to the peptide corresponding to amino acids 195-222 (SEQ ID NO: 1), as well as the characteristics described hereinbelow, advantageously allows the recruitment of effector cells. In this regard, an “effector cell” is a cell that causes the destruction of the cells to which the antibody is bound (the “target cells”). More specifically, on their surfaces, the effector cells express a receptor for the Fc fragment of the antibodies. Furthermore, the term “recruitment” refers to the ability of the antibody according to the invention to bind cells that are capable of causing the destruction of the target cells. This destruction may consist of lysis, i.e. a destruction of the target cells with release of their contents. The peptide that the antibody according to the invention can bind (the “target peptide”) is located in the binding region of the LDLs (the natural ligand to LDL-R), such that the antibody according to the invention is a good competitor of LDLs, and thus exhibits an affinity for LDL-R that is comparable to that of the natural LDL-R ligand.

Through this bond, the antibody according to the invention allows the recruitment of cells that can cause the destruction of cells to which the polypeptide according to the invention is bound, i.e. cells that express LDL-R on their surfaces (the “target cells”).

The term “bond” refers to the binding of the polypeptide to the target peptide, as well as the binding of the cells that can cause the destruction of the target cells.

The effector cells may be NK (Natural Killer) cells. They may also be macrophages, neutrophils, T4 lymphocytes, T8 lymphocytes, or eosinophils. On their surfaces, these cells express receptors for the Fc fragment of the polypeptides according to the invention. These antibodies or polypeptides bind to the target cell through their variable fragment, and bind to the effector cells through their constant fragment. This antibody-dependent relationship between the target cells and the effector cells causes the lysis of the target cells via a mechanism of the ADCC (Antibody-Dependent Cellular Cytotoxicity) type.

The target cells according to the invention are advantageously tumoural cells.

The antibody according to the invention advantageously allows the destruction of cancer cells (the “target cells”). In fact, the recruitment of effector cells entails the destruction of the cells to which the antibody according to the invention is bound. Yet, studies have demonstrated a correlation between the increase in the LDL-R expression level by the cells and certain cancers. In fact, it has been found that patients with certain cancers suffer from hypocholesterolemia. This hypocholesterolemia is the result of overuse of cholesterol by cancer cells. For their survival, the latter induce an increase in the expression level of the LDL receptor (LDL-R) within the tumoural organs (Henricksson et al., 1989). These include, particularly, prostate, breast, liver, pancreatic, ovarian, colon, lung, and stomach cancers, as well as leukemias.

Thus, the cancer cells that overexpress LDL-R will be preferred targets of the antibody according to the invention.

Therefore, the object of the invention is a monoclonal antibody raised against the human LDL (Low Density Lipoprotein) receptor, binding to the peptide corresponding to amino acids 195-222 (SEQ ID NO: 1) in the peptide sequence for the human LDL receptor.

Advantageously, at least one CDR (Complementarity-Determining Region) of each of the light chains of the antibody according to the invention has a peptide sequence that is at least 70% identical to a sequence selected from among the following sequences: SEQ ID NO: 2, SEQ ID NO: 3, SEQ ID NO: 4, SEQ ID NO: 18, SEQ ID NO: 19, SEQ ID NO: 20, and at least one CDR of each of the heavy chains of the antibody according to the invention has a peptide sequence that is at least 70% identical to a sequence selected from among the following sequences: SEQ ID NO: 5, SEQ ID NO: 6, SEQ ID NO: 7, SEQ ID NO: 21, SEQ ID NO: 22, SEQ ID NO: 23.

The CDRs in question are the CDR1 and/or CDR2 and/or CDR3 CDRs.

The sequences SEQ ID NO: 2, SEQ ID NO: 3, SEQ ID NO: 4, SEQ ID NO: 5, SEQ ID NO: 6, and SEQ ID NO: 7 are defined according to Kabat [Kabat et al. “Sequences of Proteins of Immunological Interest”, NIH Publication, 91-3242 (1991)].

The sequences SEQ ID NO: 18, SEQ ID NO: 19, SEQ ID NO: 20, SEQ ID NO: 21, SEQ ID NO: 22, and SEQ ID NO: 23 are defined according to the IMGT (International ImMunoGeneTics Database) analysis [Lefranc, M. P. et al. Dev. Comp. Immunol. 27, 55-77 (2003)]. This definition, which differs from that of Kabat—which is based solely on the analysis of variability of the sequences—takes into consideration and combines the characterisation of the hypervariable loops [Chothia C. and Lesk A. M. J. Mol. Biol. 196: 901-17 (1987)] and the crystallographic analysis of the antibody structures.

In a particularly advantageous manner, identity to each of the above-mentioned sequences is at least 70%, preferably at least 80%, 90%, 95%, or 99%, and even more preferably 100%. The identity percentage is calculated by aligning the 2 sequences to be compared and by counting the number of positions that have one identical amino acid, which number is then divided by the total number of amino acids in the sequence. In any event, these sequence differences do not in any way affect the affinity of the monoclonal antibody for its target, or its ability to recruit immune effector cells.

In a particularly advantageous manner, each CDR of each of the light chains of the antibody according to the invention has a peptide sequence that is at least 70% identical to the following sequences: SEQ ID NO: 2 or SEQ ID NO: 18; SEQ ID NO: 3 or SEQ ID NO: 19; SEQ ID NO: 4 or SEQ ID NO: 20, respectively, and each CDR of each of the heavy chains of the antibody according to the invention has a peptide sequence that is at least 70% identical to the following sequences: SEQ ID NO: 5 or SEQ ID NO: 21; SEQ ID NO: 6 or SEQ ID NO: 22; SEQ ID NO: 7 or SEQ ID NO: 23, respectively. Thus, the CDR1 region of each of the light chains of the antibody according to the invention has a peptide sequence that is at least 70% identical to sequence SEQ ID NO: 2 or sequence SEQ ID NO: 18; the CDR2 region of each of the light chains of the antibody according to the invention has a peptide sequence that is at least 70% identical to sequence SEQ ID NO: 3 or sequence SEQ ID NO: 19; the CDR3 region of each of the light chains of the antibody according to the invention has a peptide sequence that is at least 70% identical to sequence SEQ ID NO: 4 or sequence SEQ ID NO: 20; and the CDR1 region of each of the heavy chains of the antibody according to the invention has peptide sequence that is at least 70% identical to sequence SEQ ID NO: 5 or sequence SEQ ID NO: 21; the CDR2 region of each of the heavy chains of the antibody according to the invention has peptide sequence that is at least 70% identical to sequence SEQ ID NO: 6 or sequence SEQ ID NO: 22; and the CDR3 region of each of the heavy chains of the antibody according to the invention has peptide sequence that is at least 70% identical to sequence SEQ ID NO: 7 or sequence SEQ ID NO: 23. In a particularly advantageous manner, identity to each of the above-mentioned sequences is at least 70%, preferably at least 80%, 90%, 95%, or 99%, and even more preferably 100%.

Advantageously, the variable region of each of the light chains of the antibody according to the invention is coded by a nucleic acid sequence that is at least 70% identical to the nucleic acid sequence SEQ ID NO: 8, and the variable region of each of the heavy chains of the antibody according to the invention is coded by a nucleic acid sequence that is at least 70% identical to the nucleic acid sequence SEQ ID NO: 9.

In a particularly advantageous manner, identity to each of the above-mentioned sequences is at least 70%, preferably at least 80%, and even more preferably 95% or 99%. The identity percentage is calculated by aligning the 2 sequences to be compared and by counting the number of positions that have one identical nucleotide, which number is then divided by the total number of nucleotides in the sequence. The degeneracy of the genetic code may be the reason why a single amino acid can be coded by multiple triplets of different nucleotides. In any event, these sequence differences do not in any way affect the affinity of the monoclonal antibody for its target, or its ability to recruit immune effector cells.

Preferably, the variable region of each of the light chains of the antibody according to the invention is coded by the nucleic acid sequence SEQ ID NO: 8, and the variable region of each of its heavy chains is coded by the nucleic acid sequence SEQ ID NO: 9.

Advantageously, the variable region of each of the light chains of the antibody according to the invention is at least 70% identical to the amino-acid sequence SEQ ID NO: 10, and the variable region of each of its heavy chains is at least 70% identical to the amino-acid sequence SEQ ID NO: 11. In a particularly advantageous manner, identity to each of the above-mentioned sequences is at least 70%, preferably at least 80%, and even more preferably 95% or 99%. The identity percentage is calculated by aligning the 2 sequences to be compared and by counting the number of positions that have an identical amino acid, which number is then divided by the total number of amino acids in the sequence.

Preferably, the variable region of each of the light chains of the antibody according to the invention has the peptide sequence SEQ ID NO: 10, and the variable region of each of the heavy chains of the antibody according to the invention has the peptide sequence SEQ ID NO: 11. The peptide sequence SEQ ID NO: 10 is the peptide sequence deduced from the nucleotide sequence SEQ ID NO: 8, and the peptide sequence SEQ ID NO: 11 is the peptide sequence deduced from the nucleotide sequence SEQ ID NO: 9.

The antibody according to the invention also encompasses any modified antibodies that exhibit the characteristics of the invention, in which one or more amino acids have been added, substituted, or deleted. Such an addition, substitution, or deletion may be located at any position in the molecule. In the case in which multiple amino acids have been added, substituted, or deleted, any combination of additions, substitutions, or deletions may be contemplated. Such alterations in the sequence of the variable regions of the antibody according to the invention may be made in order to increase the number of residues that can come into contact with the target peptide.

Advantageously, an antibody according to the invention may be (i.e. it may consist of) an F(ab′)2 fragment, an Fab′ fragment, an Fab fragment, a CDR, or any modified version of any one of these fragments or of this region.

The antibody according to the invention is advantageously a murine antibody. This murine antibody is advantageously an IgG1κ antibody. Such an antibody may be produced through the immunisation of an animal—specifically, a mouse—with the peptide corresponding to amino acids 195-222 (SEQ ID NO: 1), or with any other human LDL-R peptide located in the LDL-binding region. Methods for producing antibodies are known to those skilled in the art. According to one particular method for producing monoclonal antibodies raised against the peptide corresponding to amino acids 195-222 (SEQ ID NO: 1) of LDL-R, the peptide corresponding to amino acids 195-222 (SEQ ID NO: 1) of the LDL-R sequence can be injected intraperitoneally into BALB/c mice in the presence of Freund's adjuvant. Multiple immunisation boosters are performed in the presence of incomplete Freund's adjuvant. The immune response of the mice is monitored through blood samples via ELISA against the SEQ ID NO: 1 peptide. Hybridomas are obtained from the fusion of spleen cells from the immunised mice with mouse myeloma cells in the presence of PEG (polyethylene glycol). The cells are then cultured, and their response against the SEQ ID NO: 1 peptide is tested via ELISA.

The antibody according to the invention is advantageously a chimeric, humanised, or human antibody.

The antibody according to the invention is preferably chimeric.

The term “chimeric antibody” refers to an antibody in which the variable regions of the light and heavy chains belong to a different species than that of the constant regions of the light and heavy chains. Thus, the antibody according to the invention further has murine variable regions and constant regions that belong to a non-murine species. In this regard, all of the families and species of non-murine mammals may be used, including, in particular, humans, monkeys, murine (except mice), porcine, bovine, equine, feline, and canine, for example, as well as birds. Even more preferably, the constant regions of each of the light chains and each of the heavy chains of the antibody according to the invention are human constant regions. This preferred embodiment of the invention makes it possible to reduce the immunogenicity of the antibody in humans, and thereby also makes it possible to improve its efficacy when administered to humans.

In a preferred embodiment of the invention, the constant region of each of the light chains of the antibody according to the invention is a K-type region. Any allotype is suitable for the embodiment of the invention, for example, Km(1), Km(1, 2), Km(1, 2, 3), or Km(3).

In another embodiment of the invention, the constant region of each of the light chains of the antibody according to the invention is a λ-type region.

In one particular aspect of the invention, and specifically when the constant regions of each of the light chains and each of the heavy chains of the antibody according to the invention are human regions, the constant region of each of the heavy chains of the antibody is a γ-type region. According to this alternative, the constant region of each of the heavy chains of the antibody may be a γ1, γ2, or γ3-type region—which three types of constant regions have the particular characteristic of binding the human complement—, or even a γ4-type region. The antibodies in which the constant region of each of the heavy chains is a γ-type region belong to the IgG class. G-type immunoglobulins (IgGs) are heterodimers consisting of 2 heavy chains and 2 light chains, linked to each other by disulfide bonds. At the N-terminus, each chain consists of a variable region or domain (coded by the rearranged V-J genes for the light chains and by the rearranged V-D-J genes for the heavy chain) that is specific for the antigen against which the antibody is raised; and, at the C-terminus, each chain consists of a constant region that consists of a single CL domain for the light chain, or of 3 domains (CH₁, CH₂, and CH₃) for the heavy chain. The association of the variable domains and of the CH₁ and CL domains of the heavy and light chains forms the Fab parts, which are connected to the Fc region by a very flexible hinge region that allows each Fab to bind to its antigenic target, while the Fc region, which is the mediator of the effector properties of the antibody, remains accessible to the effector molecules, such as the FcγR receptors and C1q. The Fc region, which consists of 2 globular domains (CH₂ and CH₃), is glycosylated at the CH₂ domain, with the presence, on each of the 2 chains, of a biantennary N-glycan linked to the Asn 297 residue.

The constant region of each of the heavy chains of the antibody is preferably a γ1-type region, because such an antibody displays an ability to exhibit ADCC (Antibody-Dependent Cellular Cytotoxicity) activity in the largest number of individuals (humans). In this regard, any allotype—for example, G1m(3), G1m(1, 2, 17), G1m(1, 17), or G1m(1, 3)—is suitable for the implementation of the invention.

The chimeric antibodies according to the invention can be constructed using the standard recombinant DNA techniques that are well known to those skilled in the art, and more specifically, using the chimeric antibody construction techniques described, for example, in Morrison et al. Proc. Natl. Acad. Sci. USA 81: 6851-55 (1984), in which the recombinant DNA technique is used to replace the constant region of a heavy chain and/or the constant region of a light chain, in an antibody obtained from a non-human mammal, with the corresponding regions in a human immunoglobulin. Such antibodies and their preparation have also been described in patent publication EP 173 494, in document Neuberger, M. S. et al. Nature 312(5995): 604-8 (1985), as well as in document EP 125 023, for example. Methods for generating chimeric antibodies are widely available to those skilled in the art. For example, the heavy and light chains of the antibody may be expressed separately by using a vector for each chain, or else may be integrated into a single vector.

An expression vector is a nucleic acid molecule into which the murine nucleic acid sequence that codes for the variable domain of each of the heavy or light chains of the antibody and the nucleic acid sequence, which is preferably human, that codes for the constant region of each of the heavy or light chains of the antibody have been inserted, in order to introduce and maintain them in a host cell. It allows the expression of these foreign nucleic acid fragments in the host cell because it contains sequences (a promoter, a polyadenylation sequence, and a selection gene) that are essential to this expression. The vector may consist, for example, of a plasmid, an adenovirus, a retrovirus, or a bacteriophage, and the host cell may be any mammalian cell, for example, SP2/0, YB2/0, IR983F, Namalwa human myeloma, PERC6, the CHO lines, particularly CHO-K-1, CHO-Lec10, CHO-Lec1, CHO-Lec13, CHO Pro-5, CHO dhfr-, Wil-2, Jurkat, Vero, Molt-4, COS-7, 293-HEK, BHK, K6H6, NS0, SP2/0-Ag14, and P3X63Ag8.653.

For the construction of expression vectors for the chimeric antibodies according to the invention, appropriate synthetic signal sequences and restriction sites may be fused to the variable regions during PCR amplification reactions. The variable regions are then combined with the constant regions of an antibody, preferably a human IgG1. The genes constructed in this way are cloned under the control of a promoter (for example, the RSV promoter) and upstream of a polyadenylation site, using two separate vectors (one for each chain). The vectors are also provided with selection genes, known to those skilled in the art, such as, for example, the dhfr gene or the neomycin resistance gene.

The chimeric antibodies according to the invention can be produced by co-transfection, into a host cell, of the expression vector of the light chain and the expression vector of the heavy chain, using a method that is well known to those skilled in the art (for example, co-precipitation with calcium phosphate, electroporation, microinjection, etc.). At the end of the transfection, the cells can be placed in a selective medium, for example, in an RPMI medium (Invitrogen, ref. 21875-034) containing 5% dialysed serum (Invitrogen, ref. 10603-017), 500 μg/mL G418 (Invitrogen, ref. 10131-027), and 25 nM methotrexate (Sigma, ref. M8407). The supernatants from the resistant transfection wells are screened for the presence of chimeric immunoglobulin (Ig) via an ELISA assay that is specific for human Ig sequences. The transfectants producing the most antibodies are amplified, and their supernatants are assayed again via ELISA in order to estimate their productivity and select the 3 best producers for limiting-dilution cloning (40 cells/plate).

The term “humanised antibody” refers to an antibody that contains CDRs derived from an antibody of non-human origin, with the other parts of the antibody molecule being derived from one (or more) human antibodies. Such antibodies can be prepared according to CDR-grafting methods that are well known to those skilled in the art [U.S. Pat. Nos. 5,225,539 and 6,180,370; Jones et al. Nature 321(6069): 522-5 (1986); Verhoeyen et al. Bioassays 8(2): 74-8 (1988); Riechmann et al. Nature 332: 323-7 (1988); Queen C. et al. Proc. Natl. Acad. Sci. USA 86(24): 10029-33 (1989); Lewis A. P. and Crowe J. S. Gene 101(2): 297-302 (1991); Daugherty B. L. et al. Nucleic Acids Res. 19(9): 2471-6 (1991); Carter et al. Proc. Natl. Acad. Sci. USA 89: 4285 (1992); Singer et al. J. Immunol. 150(7): 2844-57 (1993); and Presta et al. J. Immunol. 151: 2623 (1993)]. The selection of the human variable domains to be grafted for the production of humanised antibodies is important in order to reduce the immunogenicity of the antibody without altering its affinity for its target. In one method for producing a humanised antibody, the sequence of the variable domain of a murine antibody is compared against a library of known sequences of human variable regions, and the human variable sequence that is closest to the murine sequence is selected as the FR region of the humanised antibody [Riechmann et al. Nature 332: 323-7 (1988); Queen C. et al. Proc. Natl. Acad. Sci. USA 86(24): 10029-33 (1989); and Sims et al. J. Immunol. 151: 2296 (1993)]. Another method for selecting human FR regions consists of comparing the sequence of each sub-region of the murine FR sequence (FR1, FR2, FR3, and FR4) against a library of known human FR sequences, in order to select, for each FR region, the human FR sequence that is closest to the murine sequence [U.S. patent publication 2003/0040606; Singer et al. J. Immunol. 150(7): 2844-57 (1993); Sato K. et al. Mol. Immunol. 31(5): 371-81 (1994); and Leung S. O. et al. Mol. Immunol. 32(17-18): 1413-27 (1995)]. Another method uses a particular FR region derived from a consensus sequence of all of the human antibodies in a particular heavy-chain or light-chain sub-group [Sato K. et al. Mol. Immunol. 31(5): 371-81 (1994)]. In most cases, the CDR grafting is completed through the mutation of certain key residues located in the human FRs, in order to retain the good affinity of the humanised antibody for its target (Holmes M. A. and Foote J. J. Immunol. 158(5): 2192-201 (1997)].

The humanised antibodies according to the invention are preferred for use in in vitro diagnostic methods, or in in vivo prophylactic and/or therapeutic methods.

The antibody according to the invention, as chimerised or humanised in this way, has the advantage of being better tolerated by the human body and of being at least as effective as the murine antibody. In a particularly advantageous manner, the antibody chimerised or humanised in this way is 2 times more cytotoxic than the corresponding murine antibody. In a even more advantageous manner, the antibody chimerised or humanised in this way is 10 times, or even 100 times, or preferably, more than 100 times more cytotoxic than the corresponding murine antibody.

The term “human antibody” should be understood as denoting an antibody in which each region is derived from a human antibody. Such antibodies may be derived from transgenic mice carrying human antibody genes, or from human cells [Jakobovits et al. Curr. Opin. Biotechnol. 6(5): 561-6 (October 1995); Lonberg N. and Huszar D. Internal Review of Immunology 13: 65-93 (1995); and Tomizuka K. et al. Proc. Natl. Acad. Sci. USA 97(2): 722-727 (2000)].

The antibody according to the invention is advantageously coupled to a toxin. This toxin is, for example, diphtheria toxin or ricin. The bond between the antibody according to the invention and the toxin is strong enough to prevent the systemic release of the toxin, and is also sufficiently labile to allow the toxin to be released into the target cells.

In another aspect of the invention, the antibody is coupled to a radioisotope. The presence of the radioisotope substantially increases the cytotoxicity. Two isotopes are mainly used: iodine-131 (a β and γ emitter), whose half-life is relatively long (8 days) and that has a tumouricidal effect over an area of approximately 1 mm around the tumoural cell that bound the antibody according to the invention. Iodine-131 has the advantage of making imaging possible, but requires compliance with radioprotection measures. Yttrium-90 (a β emitter), whose half-life is shorter (2.5 days), has tumouricidal effects over a distance of 5 mm.

The antibody according to the invention advantageously allows the recruitment of immune effector cells. In fact, the antibody according to the invention is a good LDL competitor: its affinity for LDL-R is comparable to that of the natural LDL-R ligand.

Thanks to its good specificity and sensitivity, this antibody is a tool that can be used to mediate ADCC (Antibody-Dependent Cellular Cytotoxicity) reactions. In fact, the antibody according to the invention can be modified so as to induce ADCC, for example, by being chimerised or humanised. The antibody according to the invention allows the recruitment of immune effector cells. For the purposes of the invention, the term “immune effector cell” should be understood as referring to a cell that causes the destruction of the cells (the “target cells”) to which the antibody according to the invention is bound. More specifically, on their surfaces, the effector cells express a receptor for the Fc region of the antibodies. For example, the effector cells are NK (Natural Killer) cells. They may also be macrophages, neutrophils, T4 lymphocytes, T8 lymphocytes, or eosinophils. On their surfaces, these cells have receptors for the Fc region of the antibodies according to the invention.

Furthermore, the term “recruitment” refers to the ability of the polypeptide according to the invention to bind cells that are capable of causing the destruction of the target cells. This destruction may consist of lysis, i.e. a destruction of the target cells with release of their contents.

The antibody according to the invention advantageously allows the destruction of cancer cells. In fact, the recruitment of effector cells by the antibody according to the invention entails the destruction of the cells (target cells) to which the antibody is bound. Thus, cancer cells that overexpress LDL-R will be preferred targets of the antibody according to the invention. Thus, the lysed cells will quasi-specifically consist of cancer cells, because healthy cells do not overexpress LDL-R, or do so only to a limited extent, and thus are preserved.

In one particular embodiment, the antibody is produced in the SP2/0-Ag14 mouse cell line (ATCC CRL-1581).

One preferred antibody according to the invention is the 12G4 antibody produced by the H12G4 hybridoma (deposited with the CNCM under No. I-3487). The variable region of each of the light chains of the monoclonal antibody produced by the H12G4 hybridoma is coded by the nucleic acid sequence SEQ ID NO: 8, and the variable region of each of the heavy chains of the monoclonal antibody produced by the H12G4 hybridoma is coded by the nucleic acid sequence SEQ ID NO: 9.

One particular object of the invention relates to a monoclonal antibody that binds to LDL-R and allows the recruitment of effector cells. This antibody is the 12G4 antibody, or any chimeric, humanised, or human antibody that contains the variable parts of the 12G4 antibody.

Another object of the invention relates to a stable cell line that produces an antibody according to the invention as described hereinabove.

The stable cell line according to the invention is advantageously selected from among the group consisting of: SP2/0, YB2/0 (the YB2/3HL.P2.G11.16Ag.20 cell, deposited with the American Type Culture Collection under ATCC No. CRL-1662), SP2/0-AG14 (ATCC CRL-1581), IR983F, Namalwa human myeloma, PERC6, the CHO lines, particularly CHO-K-1, CHO-Lec10, CHO-Lec1, CHO-Lec13, CHO Pro-5, CHO dhfr-, Wil-2, Jurkat, Vero, Molt-4, COS-7, 293-HEK, BHK, K6H6, NS0, SP2/0-Ag14, and P3X63Ag8.653.

Another object of the invention relates to the H12G4 hybridoma deposited with the Collection Nationale de Cultures de Microorganismes [National Microorganism Culture Collection] (CNCM, Institut Pasteur, 25 rue du Docteur Roux, F-75724 Paris Cedex 15), under CNCM registration No. I-3487.

Another object of the invention relates to a DNA fragment with sequence SEQ ID NO: 9 that codes for the variable region of the heavy chain of an antibody according to the invention. This DNA fragment may be used in the manufacture of a polypeptide that binds to the peptide corresponding to amino acids 195-222 (SEQ ID NO: 1) in the peptide sequence of the human LDL receptor, which polypeptide may be an antibody.

Another object of the invention relates to a DNA fragment with sequence SEQ ID NO: 8 that codes for the variable region of the light chain of an antibody according to the invention. Similarly, this DNA fragment may be used in the manufacture of a polypeptide that binds to the peptide corresponding to amino acids 195-222 (SEQ ID NO: 1) in the peptide sequence of the human LDL receptor, which polypeptide may be an antibody.

Another object of the invention relates to an expression vector that includes at least one DNA fragment selected from among the fragments having SEQ ID NO: 9 and SEQ ID NO: 8.

Another object of the invention consists of the peptide corresponding to amino acids 195-222 (SEQ ID NO: 1) in the peptide sequence of the human LDL receptor.

Another object of the invention is the use of an antibody according to the invention to activate, in vitro or in vivo, the FcγRIII receptors of immune effector cells. In fact, the antibodies according to the invention can be used because of their ability to activate, through their Fc region, the FcγRIIIA receptor. This is of significant interest because this receptor is expressed on the surface of cells known as “effector cells”: binding of the Fc region of the antibody to its receptor carried by the effector cell causes the activation of the FcγRIIIA and the destruction of the target cells. The effector cells are, for example, NK (Natural Killer) cells, macrophages, neutrophils, CD8 lymphocytes, Tyδ lymphocytes, NKT cells, eosinophils, basophils, or mastocytes.

Another specific object of the invention is an antibody, as described hereinabove, intended for use as a drug.

In one particular aspect of the invention, the antibody that is used is bound to the human LDL receptor, and allows the recruitment of effector cells. This cytotoxic antibody can advantageously bind to all or part of the extracellular region of the LDL receptor; that is, it is capable of binding to the LDL-binding region (corresponding to amino acids 1 to 322) or to the region outside the LDL-binding region (corresponding to LDL-R amino acids 322-768). In this regard, the antibody according to the invention, which binds to the peptide corresponding to amino acids 195-222 (SEQ ID NO: 1) in the peptide sequence of the LDL receptor, is one specific embodiment of this aspect of the invention.

More specifically, one object of the invention is the use of an antibody, as described hereinabove, in the manufacture of a drug. This cytotoxic antibody can advantageously bind to all or part of the extracellular region of the human LDL receptor; that is, it is capable of binding to the LDL-binding region (corresponding to amino acids 1 to 322) or to the region outside the LDL-binding region (corresponding to LDL-R amino acids 322-768). In this regard, the antibody according to the invention, which binds to the peptide corresponding to amino acids 195-222 (SEQ ID NO: 1) in the peptide sequence of the human LDL receptor, is one specific embodiment of this object of the invention.

Another object of the invention is the use of an antibody, as described hereinabove—i.e. having the ability to bind to all or part of the extracellular region of the LDL receptor, and advantageously to the peptide corresponding to amino acids 195-222 (SEQ ID NO: 1)—in the manufacture of a drug intended for the treatment of cancer. In fact, the antibody according to the invention specifically targets LDL-R. In this regard, the antibody according to the invention, when bound to this receptor, causes a lysis reaction in the target cancer cells, specifically via ADCC against the target cancer cells, and enables their lysis. Thus, the lysed cells will quasi-specifically consist of cancer cells, because healthy cells do not overexpress LDL-R, or do so only to a limited extent, and thus are preserved.

Advantageously, the cancers treated using the antibody according to the invention are cancers in which the LDL receptor is overexpressed on the surface of the cancer cells, in comparison with the corresponding healthy cells.

In a particularly advantageous manner, the treated cancer is prostate, breast, liver, pancreatic, stomach, ovarian, colon, or lung cancer, or leukemias, in which an increase in the density of LDL receptors is observed on the membrane surface of the cancer cells. The target cancer cells may be lysed by the effector cells recruited during the ADCC reaction, with the healthy cells being preserved because they do not overexpress LDL-R.

In a particularly advantageous manner, the antibody according to the invention is used in the preparation of a drug intended for the treatment of cancers, including acute myeloid leukemia, acute monocytic leukemias, myelomonocytic leukemias, chronic myeloid leukemia in blastic crisis, lymphoid leukemias, chronic lymphoid leukemias, solid tumours such as epidermoid cervical cancer, endometrial adenocarcinoma, gastric carcinoma, hepatocellular carcinoma, choriocarcinoma, and brain tumours.

Another object of the invention relates to a pharmaceutical composition that includes an antibody according to the invention, as described hereinabove, and a pharmaceutically acceptable excipient and/or carrier. The purpose of this pharmaceutical composition is to target the cancer cells, specifically the ones that overexpress LDL-R. Because these cancer cells express, on their surfaces, an amount of LDL receptors that is greater than the amount of receptors expressed by the healthy cells, the drug prepared in this manner will be preferentially bound by the cancer cells.

The excipient may be any solution, such as a saline, isotonic, or buffered solution, etc., as well as any suspension, gel, or powder, etc. that is compatible with pharmaceutical use and known to those skilled in the art. The compositions according to the invention may also contain one or more agents or carriers selected from among dispersants, solubilisers, stabilisers, surfactants, preservatives, etc. The compositions according to the invention may also include other agents or active ingredients.

Furthermore, the compositions may be administered in different ways and in different forms. Administration may consist of any traditional route for this type of therapeutic approach, such as, specifically, the systemic route, including, in particular, intravenous, intradermal, intratumoural, subcutaneous, intraperitoneal, intramuscular, or intraarterial injection, etc. For example, reference may be made to intratumoural injection, or injection in an area near the tumour or irrigating the tumour.

Dosages may vary depending on the number of administrations, the association with other active ingredients, the stage of progress of the disease condition, etc.

Another object of the invention is the use of the antibody according to the invention in immunohistochemical analyses of cancerous, healthy, or cirrhotic tissues, or in Western-Blot or ELISA analyses, or in in vivo quantification tests.

Other aspects and advantages of the invention will be described in the following examples, which must be considered as illustrative and do not limit the scope of the invention.

### FIGURE LEGENDS

FIG. 1: Screening of the LDL-R expression level in cancer cell lines (results expressed as arbitrary fluorescence units).

FIG. 2: LDL-Dil binding to HepG2 cells (results expressed as the percentage of fluorescent cells).

FIG. 3: Screening of breast-cancer cell lines for LDL-R expression (results expressed as the percentage of fluorescent cells).

FIG. 4A: Binding of anti-LDL-R 12G4 antibodies to A549 cells (results expressed as the percentage of fluorescent cells). The anti-LDL-R 12G4 antibodies are produced by the H12G4 hybridoma and are raised against the peptide corresponding to amino acids 195-222 in the LDL receptor sequence (SEQ ID NO: 1).

FIG. 4B: Binding of anti-LDL-R 12G4 antibodies to A549 cells (results expressed as mean fluorescence).

FIG. 5A: Binding of anti-LDL-R 12G4 antibodies to MDA-MB-231 cells (results expressed as the percentage of fluorescent cells).

FIG. 5B: Binding of anti-LDL-R 12G4 antibodies to MDA-MB-231 cells (results expressed as mean fluorescence).

FIG. 6A: Cross-reactivity of anti-LDL-R 12G4 antibodies on C2C12, CHO-K1, and YB2/0 cells (results expressed as the percentage of fluorescent cells).

FIG. 6B: Cross-reactivity of anti-LDL-R 12G4 antibodies on C2C12, CHO-K1, and YB2/0 cells (results expressed as mean fluorescence).

FIG. 7: Competition of anti-LDL-R 12G4 antibodies with LDL, on A549 cells (results expressed as mean fluorescence).

FIG. 8: Internalisation kinetics of anti-LDL-R 12G4 antibodies with LDL, on A549 cells (results expressed as the internalisation percentage).

FIG. 9: Western-Blot characterisation of the 12G4 antibody.

### EXAMPLES

### Example 1

### Production, Selection, and Characterisation of Monoclonal Antibodies Raised Against the Peptide Corresponding to the Sequence of Amino Acids 195-222 in the Human LDL Receptor Sequence (SEQ ID NO: 1)

### Selection of the Appropriate Peptide Sequence

The peptide fragment corresponding to sequence SEQ ID NO: 1 (corresponding to amino acids 195-222 in the human LDL receptor sequence) located in the LDL-binding region was synthesized. The selected sequence SEQ ID NO: 1 was modified (by replacing the cysteine residues with serine residues) in order to avoid the formation of disulfide bonds in the event of an oxidation of the thiol group of the cysteine residues; the corresponding sequence is sequence SEQ ID NO: 17.

### Peptide Synthesis

The peptide was synthesized via the solid-phase synthesis method, on an ABI 433 A-model automatic synthesizer (Applied Biosystems Inc., California, U.S.A.), using a Boc/Bzl strategy on a 0.5-mmol MBHA resin.

### Mass Spectrometry

The molecular mass was determined by ion-electrospray mass spectrometry. The electrospray spectrum was obtained using an API system (Perkin-Elmer-Sciex) on an ion-electrospray single quadrupole mass spectrometer equipped with an ion spray (nebuliser-assisted electrospray) source (Sciex, Toronto, Canada).

### Production of Monoclonal Antibodies

The monoclonal antibodies raised against the peptide corresponding to sequence SEQ ID NO: 1 were produced by immunising male BALB/c mice by intraperitoneal injection of the peptide corresponding to SEQ ID NO: 17, which peptide was emulsified beforehand with an equal volume of complete Freund's adjuvant.

Three injections were then administered every two weeks, in the presence of incomplete Freund's adjuvant.

Four days after the last injection, the animals' spleens were removed, and then the cells were isolated and fused with Sp2/0-Ag14 mouse myeloma cells in the presence of a fusion agent such as polyethylene glycol. The fused cells were then incubated in a selective medium (HAT medium) that inhibits the growth of unfused malignant cells.

To check the monoclonal nature of the hybridomas, repeated limiting-dilution sub-clonings were performed. At the end of these sub-clonings, a hybridoma referred to as “H12G4”, which produces antibodies raised against the peptide corresponding to SEQ ID NO: 1, was selected. This hybridoma is a member of the IgG class, in sub-class 1.

The antibodies produced by hybridoma H12G4 were tested, via ELISA , for the secretion of a monoclonal antibody having the desired specificity, i.e. against the peptide corresponding to SEQ ID NO: 1.

The ascites were obtained from male BALB/c mice that had previously received an injection of pristane and into which 2×10⁶ cells of hybridoma H12G4 had been injected.

The monoclonal antibodies were isolated by precipitation with 27% ammonium sulfate, and then purified by affinity chromatography on Protein-A gel (HiTrap Protein-A HP columns, Amersham Bioscience, Uppsala, Sweden). The unretained proteins were washed away with buffered saline (PBS: 50 mmol/L phosphate, pH 7.2, 150 mmol/L NaCl). The elution of the monoclonal IgG immunoglobulins specific to the antibody raised against the peptide corresponding to sequence SEQ ID NO: 1 was performed using 0.2M glycine, pH 2.8. The purified antibodies were immediately dialysed against 10 mmol/L PBS, concentrated by lyophilisation, and then stored in aliquots of 0.5 to 1 mg±1% BSA at −20° C.

These antibodies will be referred to hereinbelow as the “anti-LDL-R 12G4” antibodies.

### Western-Blot Analysis (FIG. 9)

The anti-LDL-R 12G4 antibodies were tested via the Western-Blot method. Extracts of total proteins of MDA-MB-231 cells were subjected to denaturing electrophoresis on SDS-PAGE gel (10%), and then transferred to a nitrocellulose membrane and reacted with the anti-LDL-R 12G4 antibodies. The immunoreactive proteins were visualised using a peroxidase-conjugated anti-IgG monoclonal antibody (Chemicon). The development of the reaction was performed by chemiluminescence (Amersham Biosciences).

### Isotyping

The isotyping of the hybridomas was performed via ELISA, using the SBA Clonotyping System/HRP kit (SouthernBiotech) and the Isostrip Mouse Monoclonal Antibody Isotyping Test (Roche reference 1493027).

### Example 2

### Screening of the LDL-R Expression Level in Cancer Cell Lines

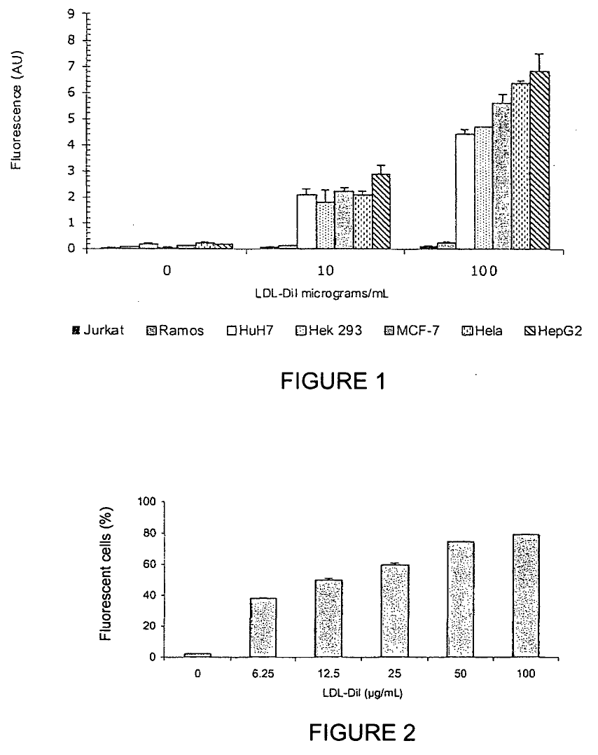

The following cancer cell lines were screened for LDL-R expression: HepG2, HeLa, MCF-7, Jurkat, Ramos, HuH7, and Hek293, by studying the binding of labelled LDLs (FIGS. 1 and 2). For this purpose, LDLs (density=1.03-1.053 g/mL) were prepared by ultracentrifuging, dialysed in a PBS buffer at a pH of 7.4, and validated via SDS-PAGE under denaturing conditions, and then labelled with fluorochrome 1,1′-dioctadecyl-3,3,3′,3′-tetramethyl-indocarbocyanide (Dil). LDL-Dils were incubated on cells at final concentrations of 0, 10, and 100 μg/mL for 3 hours at 4° C. After washing with PBS, the binding was analysed via FACS (Fluorescence-Activated Cell Sorting) cytofluorometry; that is, the fluorescence of each cell in a given population was measured individually by flow cytometry, on a FACScalibur device (Becton Dickinson). The measured parameters were the FSC (Forward Scatter), SSC (Side Scatter), and the fluorescence emitted at a wavelength of 530 nm after excitation with an argon laser at 488 nm. The results were expressed as the percentage of fluorescent cells (FIG. 1).

The results depicted in FIG. 1 show that the HepG2 and HeLa cells express LDL-R most strongly.

Furthermore, several breast-cancer cell lines were available: MCF7-ras, MDA-MB-435, and MDA-MB-231. Expression of LDL-R in these human cancer cell lines was detected by studying the binding of labelled LDLs to this cell line. For this purpose, LDLs (d=1.03-1.053) were prepared by ultracentrifuging, dialysed in a PBS buffer at a pH of 7.4, and validated via SDS-PAGE under denaturing conditions, and then labelled with fluorochrome 1,1′-dioctadecyl-3,3,3′,3′-tetramethyl-indocarbocyanide (Dil). LDL-Dils were incubated on the cells at final concentrations of 6.25, 12.5, 25, 50, and 100 μg/mL for 3 hours at 4° C. After washing with PBS, the binding was analysed via cytofluorometry (FACS) and the results were expressed as the percentage of fluorescent cells.

Thus, each line was tested for its LDL-R expression level (FIG. 2): the MCF7-ras and MDA-MB-435 cells had an LDL-R expression level equivalent to half that of the HepG2 cells. The MDA-MB-231 cells represented a homogeneous population that expressed LDL-R at a high level.

### Example 3

### In Vitro Functional Tests of Monoclonal Antibodies Raised Against the Peptide Corresponding to Sequence SEQ ID NO: 1 as Selected in Example 1

The functionality of the monoclonal antibodies raised against the peptide corresponding to sequence SEQ ID NO: 1 was evaluated by studying the binding of the antibodies to LDL-R at the cellular level (A549 cells and MDA-MB-231 cells); studying the cross-reactivity of the antibodies to LDL-R on C2C12 (mouse), CHO-K1 (hamster), and YB2/0 (rat) cells; studying the competition between these antibodies and LDLs on the LDL receptor of A549 cells; studying their internalisation kinetics; and studying the proapoptotic nature of the antibodies.

### Study of the Binding of Anti-LDL-R 12G4 Antibodies to the LDL Receptor of A549 Cells

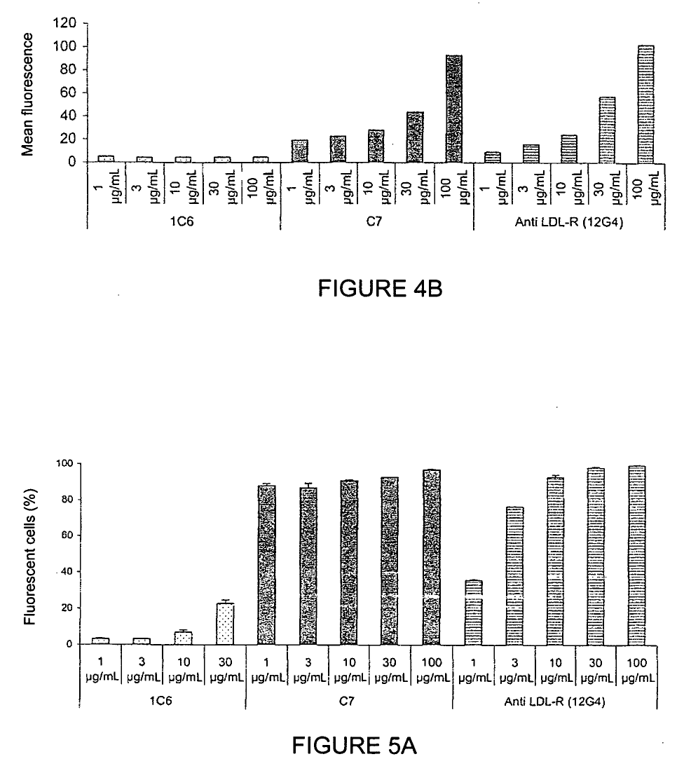

The binding of the anti-LDL-R 12G4 antibodies to LDL-R was evaluated through quantification of the labelling of A549 cells, grown in the presence of LPDS (Lipoprotein-Deficient Serum) for 24 hours, via flow cytometry (FACS). To perform this test, the anti-LDL-R 12G4 antibodies were incubated at final concentrations of 1, 3, 10, 30, and 100 μg/mL for 3 hours at 4° C. The commercial 1C6 (anti-SREBP2, IgG1, ATCC-LGC Promochem CRL-2224) and C7 (anti-LDL-R, IgG2b, ATCC CRL-1691) antibodies, prepared and incubated under the same conditions as the anti-LDL-R 12G4 antibodies, were used as negative and positive control antibodies, respectively. The detection of the bond was performed using a secondary anti-IgG-PE antibody. The results were expressed as the percentage of fluorescent cells (FIG. 4A) and as mean fluorescence (FIG. 4B).

The anti-LDL-R 12G4 antibodies and the C7 control antibody recognised the LDL receptor of the A549 cells.

### Study of the Binding of Anti-LDL-R 12G4 Antibodies to the LDL Receptor of MDA-MB-231 Cells

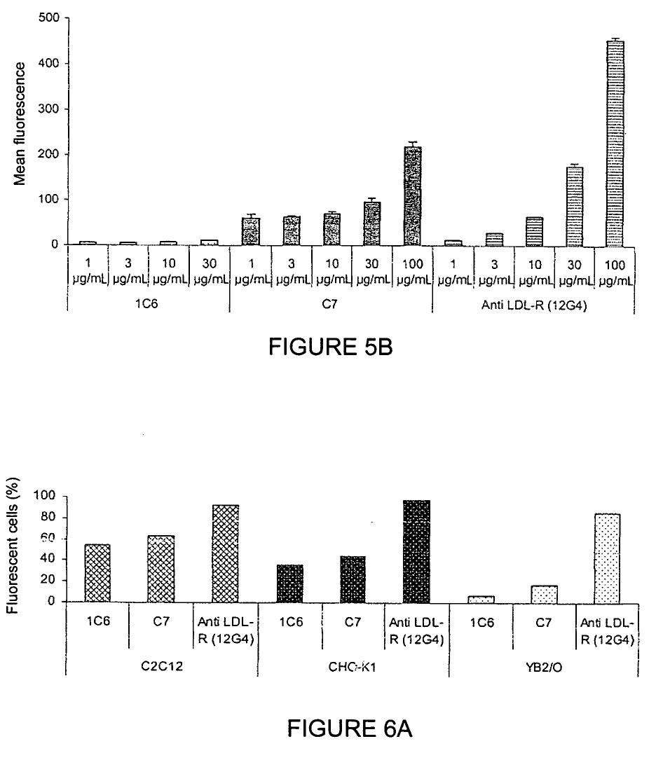

The binding of the anti-LDL-R 12G4 antibodies to the LDL receptor of MDA-MB-231 cells was evaluated according to the same protocol as described for the study of the binding of the anti-LDL-R 12G4 antibodies to the LDL receptor of the A549 cells, through quantification of the labelling of MDA-MB-231 cells grown in the presence of LPDS for 24 hours, via flow cytometry (FACS). To perform this test, the anti-LDL-R 12G4 antibodies were incubated at final concentrations of 1, 3, 10, 30, and 100 μg/mL for 3 hours at 4° C. The commercial 1C6 (anti-SREBP2, IgG1) and C7 (anti-LDL-R, IgG2b) antibodies, prepared and incubated under the same conditions as the anti-LDL-R 12G4 antibodies, were used as negative and positive control antibodies, respectively. The detection of the bond was performed using an anti-IgG-PE antibody. The results were expressed as the percentage of fluorescent cells (FIG. 5A) and as mean fluorescence (FIG. 5B).

At low antibody concentrations (1-3 μg/mL), the C7 control antibody bound to the LDL receptor of the MDA-MB-231 cells more significantly than the anti-LDL-R 12G4 antibodies do. On the other hand, at higher concentrations (10-100 μg/mL), the anti-LDL-R 12G4 antibody recognised LDL-R more significantly than the C7 antibody does.

### Study of the Cross-Reactivity of the Anti-LDL-R 12G4 Antibodies to the LDL Receptors of C2C12, CHO-K1, and YB2/0 Cells

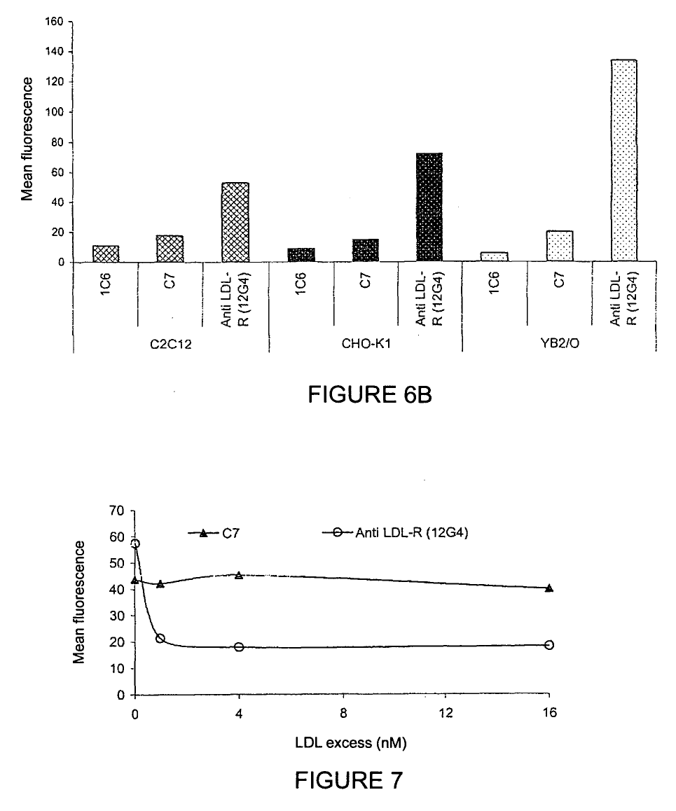

The cross-reactivity of the anti-LDL-R antibodies was tested in the mouse (C2C12 cells), in the rat (YB2/0 cells), and in the hamster (CHO-K1 cells). The anti-LDL-R 12G4 antibodies were incubated at a final concentration of 30 μg/mL for 3 hours at 4° C. on C2C12, CHO-K1, and YB2/0 cells that had been cultured beforehand under LPDS conditions for 24 hours. The commercial 1C6 (anti-SREBP2, IgG) and C7 (anti-LDL-R, IgG2b) antibodies were used as negative and positive control antibodies, respectively. The detection of the bond was performed using an anti-IgG-PE antibody. The results were expressed as the percentage of fluorescent cells (FIG. 6A) and as mean fluorescence (FIG. 6B).

Only the anti-LDL-R 12G4 antibodies cross-reacted with the mouse, the rat, and the hamster. The C7 antibody did not cross-react with the mouse or with the hamster, but did exhibit a very slight degree of cross-reactivity with the rat.

Study of the Competition of Anti-LDL-R 12G4 Antibodies with LDLs on A549 Cells

The competition of LDLs with anti-LDL-R 12G4 antibodies was studied by testing the binding of the anti-LDL-R 12G4 antibodies (at 30 μg/mL) in competition with unlabelled LDLs at increasing concentrations (1, 4, and 16 times the concentration of the antibodies, expressed as nM) to A549 cells for 3 hours at 4° C. The detection of the bond was performed using an anti-IgG-PE antibody. The binding of the antibodies to LDL-R was then analysed via FACS, and the results were expressed as mean fluorescence (FIG. 7).

This test of the competition of the antibodies with LDLs made it possible to demonstrate that binding of the C7 antibody to the LDL receptor of the A549 cells was not reduced by the addition of LDLs, at physiological concentrations, to the medium. This means that the C7 antibodies do not bind to the same site as LDLs. On the other hand, the anti-LDL-R 12G4 antibody binds less well to LDL-R in the presence of LDLs (a 60% reduction in the bond, with a fluorescence mean of 55 in the absence of LDLs, and a fluorescence mean of 20 with a 16-fold excess of LDLs). These results suggest that the anti-LDL-R 12G4 antibody binding site is the same as that of LDLs.

### Internalisation Kinetics of Anti-LDL-R 12G4 Antibodies

The internalisation kinetics of the anti-LDL-R 12G4 antibody were studied over a period of 24 hours during the incubation at 37° C. of the antibody (30 μg/mL) labelled with rhodamine (NHS-rhodamine, Pierce, ref. 46102) on A549 cells. The internalisation kinetics of the rhodamine-labelled C7 control antibody (30 μg/mL) and of LDL-Dils (30 μg/mL) were studied in parallel. After 2, 4, 6, and 24 hours of incubation, the bound but not internalised antibodies/LDL-Dils were detached using dextran sulfate and quantified via fluorimetry. The A549 cells were then lysed with soda (0.1N), and then the amount of internalised antibodies was quantified via fluorimetry. The internalisation percentage was calculated according to the following formula: fluorescence of internalised antibodies/(fluorescence of internalised antibodies+fluorescence of bound but not internalised antibodies).

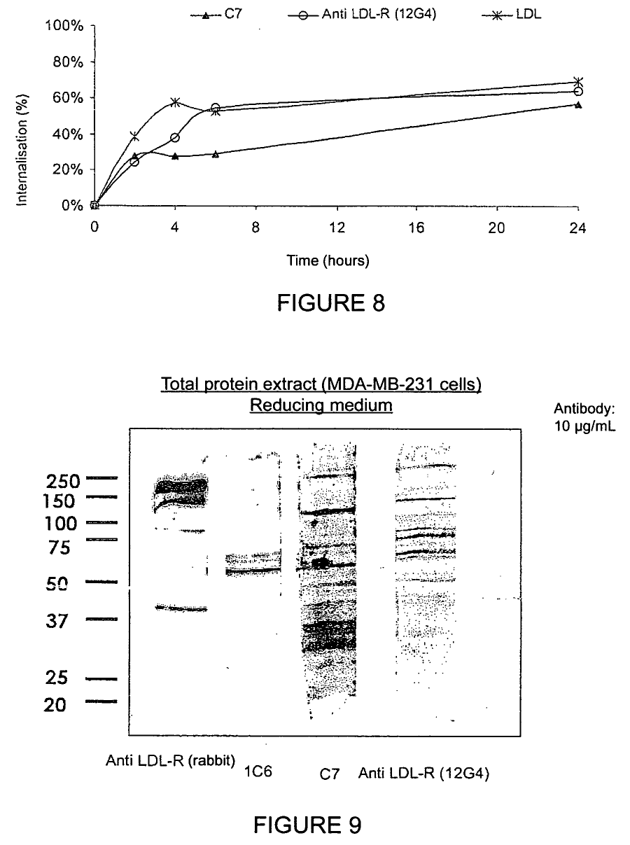

The internalisation kinetics of the anti-LDL-R 12G4 antibodies were intermediate, between those of LDLs (the most rapid kinetics, with 60% internalisation after 4 hours) and those of the C7, which was internalised a little more slowly (FIG. 8). As with LDLs, the internalisation kinetics of the antibodies were biphasic, with rapid internalisation during the first 4 to 6 hours. After 6 hours of incubation, the 3 antibodies tested exhibited the same internalisation rate, with an internalisation plateau on the order of 60% after 24 hours.

### Study of the Proapoptotic Nature of Anti-LDL-R 12G4 Antibodies

The growth of A549 cells in the presence and in the absence of anti-LDL-R 12G4 antibodies was studied through dual labelling with FITC-annexin V (which binds to the phosphatidylserine of apoptotic cells in early phase) and propidium iodide (PI, which labels only the necrotic cells whose plasma membrane has been damaged), via flow cytometry (FACS). To perform this test, the anti-LDL-R 12G4 antibodies were incubated at a final concentrations of 30 μg/mL for 16 hours (the duration of an ADCC test) at 37° C. The commercial 1C6 (anti-SREBP2, IgG1) and C7 (anti-LDL-R, IgG2b) antibodies, prepared and incubated under the same conditions as the anti-LDL-R 12G4 antibodies, were used as references. A negative control, consisting of cells without antibodies, and a positive control, consisting of cells incubated with camptothecine, were prepared in parallel.

The anti-LDL-R 12G4 antibodies did not exhibit a strongly proapoptotic effect on the A549 cells after 16 hours of incubation.

### Example 4

### In Vivo Studies

The selected animal model was a model consisting of a xenograft of human tumoural tissue on nude mice. The xenograft of tumoural cells was implanted subcutaneously.

### Selection of the Human Cancer Cell Line for the Xenograft

Screening of Cell Lines for LDL-R Expression

Several breast-cancer cell lines were available: MCF7-ras, MDA-MB-435, and MDA-MB-231; for our study, the selection of the line to be implanted depended on the LDL-R expression level. Expression of LDL-R in these human cancer cell lines was detected by studying the binding of labelled LDLs to this cell line. For this purpose, LDLs (d=1.03-1.053 g/mL) were prepared by ultracentrifuging, dialysed in a PBS buffer at a pH of 7.4, and validated via SDS-PAGE under denaturing conditions, and then labelled with fluorochrome 1,1′-dioctadecyl-3,3,3′,3′-tetramethyl-indocarbocyanide (Dil). LDL-Dils were incubated on the cells at final concentrations of 6.25, 12.5, 25, 50, and 100 μg/mL for 3 hours at 4° C. After washing with PBS, the binding was analysed via cytofluorometry (FACS) and the results were expressed as the percentage of fluorescent cells.

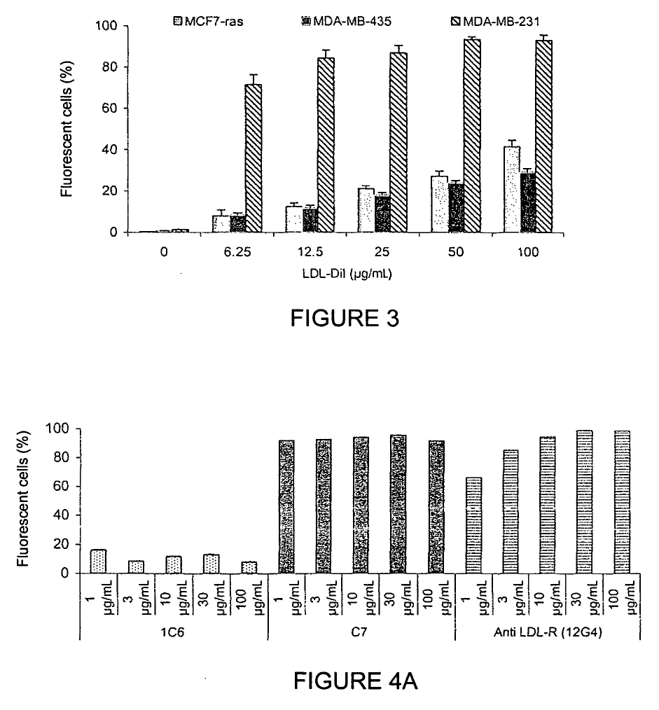

Thus, each cell line was tested for its LDL-R expression level (FIGS. 2 and 3): the MCF7-ras and MDA-MB-435 cells had an LDL-R expression level equivalent to half that of the HepG2 cells. The MDA-MB-231 cells represented a homogeneous population that expressed LDL-R at a high level. In view of these results, we selected the MDA-MB-231 cells as the line to be implanted.

Determination of the Number of Cells to be Implanted for the Xenograft

The rate of onset of the tumour as a function of the number of cells implanted in nude mice was studied. The study concentrated on the implantation of 0.5×10⁶, 10⁶, 2×10⁶ and 5×10⁶ cells.

Binding of the Anti-LDL-R 12G4 Antibody to the LDL Receptor of MDA-MB-231 Cells

The binding of the anti-LDL-R 12G4 antibody to LDL-R was studied on MDA-MB-231 cells according to the same protocol as for the study of the binding of the anti-LDL-R 12G4 antibody to the LDL receptor of HepG2 cells (Example 2), but in an indirect manner, because the antibody was not directly labelled: the antibody was incubated on MDA-MB-231 cells for 3 hours at 4° C., and then detected with an FITC-conjugated anti-IgG monoclonal antibody for FACS analysis. The results were expressed as the percentage of fluorescent cells.

Study of the Competition of the Anti-LDL-R 12G4 Antibody with LDLs on MDA-MB-231 Cells

The competition of LDLs with the anti-LDL-R 12G4 antibody was studied by testing the binding of the Dil-labelled LDLs, at 12.5 μg/mL, in competition with the anti-LDL-R 12G4 antibody at increasing concentrations (6.25, 12.5, 25, 50, and 80 μg/mL) to MDA-MB-231 cells for 3 hours at 4° C. The binding of the antibodies to LDL-R was then analysed via FACS, and the results were expressed as the percentage of fluorescent cells.

### In Vivo Protocol

For the treatment of the mice with the anti-LDL-R 12G4 antibody, the selected approach consisted of injecting the cell line and the antibody simultaneously (the Winn test). This approach made it possible to evaluate the antibody's ability to prevent the formation of the tumour.

The first group (the control group) consisted of 5 nude mice implanted with 10⁶ MDA-MB-231 cells taken up in 200 μL of a control antibody, of the same isotype as the anti-LDL-R 12G4 (IgG1) antibody that does not recognise LDL-R, with no subsequent treatment after the implantation of the cells.

The second group consisted of 5 nude mice implanted with 106 MDA-MB-231 cells taken up in 200 μL of anti-LDL-R 12G4 antibody, with no subsequent treatment after the implantation of the cells.

A “modified” approach to the Winn test was also implemented, which consisted of treating the nude mice, after the simultaneous implantation of the MDA-MB-231 cells and of the anti-LDL-R 12G4 antibody, with the anti-LDL-R 12G4 antibody, twice a week during the 4 weeks of the study (the third group). The third group consisted of 5 nude mice implanted with 10⁶ MDA-MB-231 cells taken up in 200 μL of anti-LDL-R 12G4 antibody, treated intraperitoneally with 500 μg of anti-LDL-R 12G4 antibody twice a week (with administration of the first 500 μg treatment 3 to 4 hours after the implantation of the cells).

The mice were examined every day, with measurement of the weight gain and of the size of the tumour 3 times a week. At the end of the 4 weeks of the protocol, the mice were sacrificed and the liver, heart, kidneys, and spleen were recovered and frozen at −80° C. for each of the 5 mice in each group. The mouse serums were also collected and frozen.

### Example 5

### Therapeutic Feasibility Study

The goal of the therapeutic feasibility study was to detect overexpression of LDL-R in cancer tissue in comparison with healthy tissue, in different types of cancers. For this purpose, a Western-Blot (WB) analysis of hepatocellular carcinomas was performed.

A Western-Blot analysis was performed using tissues obtained from patients with hepatocellular carcinoma (HCC) that occurred in conjunction with alcohol-induced cirrhosis (3 patients) or in conjunction with cirrhosis caused by infection with the hepatitis-C virus (15 patients). Two tissue samples were provided for each patient: (i) a sample taken from a cirrhotic but non-tumoural area, and (ii) a sample taken from tumoural tissue, obtained from the same patient (with a minimum of 70% tumoural hepatocytes).

Western Blot-type analyses were performed on these tissues, using a rabbit polyclonal anti-LDL-R antibody (Research Diagnostics Inc.). A band at 160 kDa, which corresponds to the mature form of LDL-R, was observed in both the cirrhotic and cancer tissues. In addition to this band, a band at 120 kDa, corresponding to the immature (non-glycosylated) form of LDL-R, was also present.

The quantification of the band corresponding to mature LDL-R, normalised in relation to the actin, made it possible to detect:
LDL-R overexpression in the healthy tissue of the 3 patients with HCC that occurred in conjunction with alcohol-induced cirrhosis, and in the healthy tissue of 2 of the 15 patients with HCC that occurred in conjunction with cirrhosis caused by infection with the hepatitis-C virus.
LDL-R overexpression in the cancer tissue of 7 of the 15 patients with HCC that occurred in conjunction with cirrhosis caused by infection with the hepatitis-C virus (the overexpression level among these patients was between 2- and 14-fold.)
Six patients with HCC that occurred in conjunction with cirrhosis caused by infection with the hepatitis-C virus did not exhibit any difference in terms of LDL-R expression.

### Example 6

### Study of the Antibody's Specificity

The immunohistochemical study was performed on normal human tissue microarray slides containing 108 spots corresponding to 54 different normal human tissues fixed in 10% formol. The C7 antibody was diluted to 1/10 (10 μg/mL) and the anti-LDL-R 12G4 was diluted to 1/50 (2 μg/mL). The antigenic reactivation was performed using microwaves (3 times 5 minutes at 750 watts, in a 10 mM citrate buffer, pH 6).

An absence of labelling was observed in the hematopoietic system (tonsil, spleen, and lymph node), the muscle tissue (heart and striated skeletal muscle), the skin, and the mammary tissue, regardless of the antibody employed. The central nervous system (cerebral cortex, cerebellum, central nuclei, hippocampus, and spinal cord), adrenal (cortex and medullary) glands, liver, and gall bladder were labelled by the 2 antibodies. The anti-LDL-R 12G4 antibody and the C7 antibody labelled the testes, the colon, and the pancreas, with better labelling with the anti-LDL-R 12G4 antibody for the latter two organs. Only the anti-LDL-R 12G4 antibody labelled the gastric mucosa, the basal cell layer of the buccal and exocervical mucosa, and the various renal tubules.

These results are consistent with the literature on LDL-R tissular distribution, because the adrenal glands, the liver, the central nervous system, the testes, and the kidneys have been described as tissues that strongly express LDL-R. The anti-LDL-R 12G4 antibody and the C7 antibody produce labellings of equivalent intensity, with slight higher intensity for the anti-LDL-R 12G4 antibody. Thanks to its good sensitivity and its specificity, the anti-LDL-R 12G4 antibody was selected for the immunohistochemical analysis of mammary adenocarcinomas.

### Immunohistochemical Analysis of Mammary Adenocarcinomas

LDL-R expression was studied immunohistochemically on 34 mammary adenocarcinomas and on the adjacent non-tumoural tissue with the anti-LDL-R 12G4 antibody.

The intensity of the labelling was strong, and each time labelling was observed (in 65% of the samples), only the tumoural cells were labelled, thus indicating LDL-R overexpression in cancer cells.

### Example 7

### Amplification and Sequencing of the Variable Regions of the Anti-LDL-R 12G4 Antibodies

### 1. Amplification of the VH and Vκ Regions

The total RNA of the murine 12G4 hybridoma, which produces an immunoglobulin of the IgG1κ-type, was extracted (Macherey-Nagel Nucleospin RNA kit, ref. 740609.250). The variable domains of the light chain (Vκ) and heavy chain (VH) of the 12G4 antibody were amplified using the 5′RACE (Rapid Amplification of cDNA Ends) technique (Invitrogen 5′RACE kit, ref. 18374.041), through anchoring in the murine constant κ region (Cκ) for the light chain, or in the murine G1 region for the heavy chain.

Briefly, an initial reverse-transcription phase was first performed using a primer located in the 5′ region of the murine constant Cκ or G1 regions. A poly-dC sequence was then added at the 3′ terminus of the cDNAs synthesized prior to the amplification of the Vκ and VH regions using a 5′ primer recognising the poly-dC sequence, and a 3′ primer located in the murine constant Cκ or G1 regions at the 5′ terminus of the reverse-transcription primer. To improve the specificity of the amplification, a second, “semi-nested” PCR was performed on the PCR VH product, using a 3′ primer located at the 5′ terminus of the 3′ primer of the initial PCR.

The primers used for these various stages are listed below:

1) Reverse-transcription primers
a. Murine κ-specific antisense primer

(SEQ ID NO: 12)

5′-ACT GCC ATC AAT CTT CCA CTT GAC-3′
b. Murine G1-specific antisense primer

(SEQ ID NO: 13)

5′-CTGGACAGGGATCCAGAGTTCCA-3′
2) 5′RACE PCR primers
a. Murine κ-specific antisense primer

(SEQ ID NO: 14)

```
      5′-TTGTTCAAGAAGCACACGACTGAGGCAC-3′
b. Murine G1-specific antisense primers
      First PCR primer:
```

(SEQ ID NO: 15)

```
      5′-TGTCACTGGCTCAGGGAAATAGCCCTTGAC-3′
      “Semi-nested” PCR primer:
```

(SEQ ID NO: 16)

5′-CACCATGGAGTTAGTTTGGGCAGCAGATCCA-3′

### 2. Determination of the Sequences of the VH and Vκ Regions

After amplification, the Vκ and VH sequences of the 12G4 antibody were cloned into the pCR4-TOPO plasmid (TOPO-TA-Cloning Kit for Sequencing, Invitrogen, ref. 45-0030). The plasmids from at least 3 recombinant colonies were purified, and their inserts were sequenced using the M13-uni and M13-rev universal primers.

The nucleotide sequence of the Vκ region of the 12G4 murine antibody is indicated under sequence SEQ ID NO: 8, and the deduced peptide sequence is sequence SEQ ID NO: 10. The Vκ gene belongs to the Vκ8 sub-group (Almagro J. C. et al. Immunogenetics 1998, 47: 355-363). The CDR1, CDR2, and CDR3 sequences of the Vκ region of the murine 12G4 antibody, as defined according to Kabat [Kabat et al. “Sequences of Proteins of Immunological Interest”. NIH Publication, 91-3242 (1991)], are indicated under the following sequences: SEQ ID NO: 2, SEQ ID NO: 3, and SEQ ID NO: 4, respectively. The CDR1-IMGT, CDR2-IMGT, and CDR3-IMGT sequences of the Vκ region of the murine 12G4 antibody, as defined according to the IMGT (International ImMunoGeneTics Database) analysis [Lefranc, M. P. et al. Dev. Comp. Immunol. 27, 55-77 (2003)], are indicated under the following sequences: SEQ ID NO: 18, SEQ ID NO: 19, and SEQ ID NO: 20, respectively. This definition, which differs from that of Kabat—which is based solely on the analysis of variability of the sequences—takes into consideration and combines the characterisation of the hypervariable loops [Chothia C. and Lesk A. M. J. Mol. Biol. 196: 901-17 (1987)] and the crystallographic analysis of the antibodies structures.

The nucleotide sequence of the VH region of 12G4 is sequence SEQ ID NO: 9, and the deduced peptide sequence is sequence SEQ ID NO: 11. The VH gene belongs to the VH9 sub-group (Honjo T. and Matsuda F. In: “Immunoglobulin genes”. Honjo T. and Alt F. W., eds., Academic Press, London, 1996, pp. 145-171). The CDR1, CDR2, and CDR3 sequences of the VH region of the murine 12G4 antibody, as defined according to Kabat (Kabat et al. “Sequences of Proteins of Immunological Interest”. NIH Publication, 91-3242 (1991)], are indicated under the following sequences: SEQ ID NO: 5, SEQ ID NO: 6, and SEQ ID NO: 7, respectively. The CDR1-IMGT, CDR2-IMGT, and CDR3-IMGT sequences of the VH region of the murine 12G4 antibody, as defined according to the IMGT (International ImMunoGeneTics Database) analysis [Lefranc, M. P. et al. Dev. Comp. Immunol. 27, 55-77 (2003)], are indicated under the following sequences: SEQ ID NO: 21, SEQ ID NO: 22, and SEQ ID NO: 23, respectively. This definition, which differs from that of Kabat—which is based solely on the analysis of variability of the sequences—takes into consideration and combines the characterisation of the hypervariable loops [Chothia C. and Lesk A. M. J. Mol. Biol. 196: 901-17 (1987)] and the crystallographic analysis of the antibodies structures.

### REFERENCES

Agnani G., Delpierre C. et al. (1989) “Antipeptide antibody against the human low-density-lipoprotein receptor. Characterization and cross-reactivity with bovine lymphocytes.” Biochem. J. 263(3): 753-60.
Carter P., Presta L. et al. (1992) “Humanization of an anti-p185HER2 antibody for human cancer therapy.” Proc. Natl. Acad. Sci. USA 89: 4285.
Chothia C. and Lesk A. M. (1987) “Canonical structures for the hypervariable regions of immunoglobulins.” J. Mol. Biol. 196: 901-17.
Daugherty B. L., DeMartino J. A. et al. (1991) “Polymerase chain reaction facilitates the cloning, CDR-grafting, and rapid expression of a murine monoclonal antibody directed against the CD18 component of leucocyte integrins.” Nucleic Acids Res. 19(9): 2471-6.
Gal D., Ohashi M. et al. (1981) “Low-density lipoprotein as a potential vehicle for chemotherapeutic agents and radionucleotides in the management of gynecologic neoplasms.” Am. J. Obstet. Gynecol. 139(8): 877-85.
Henriksson P., Eriksson M. et al. (1989) “Hypocholesterolaemia and increased elimination of low-density lipoproteins in metastatic cancer of the prostate.” Lancet 2(8673): 1178-80.
Holmes M. A. and Foote J. (1997) “Structural consequences of humanizing an antibody.” J. Immunol. 158(5): 2192-201.
Jones P., Dear P. et al. (1986) “Replacing the complementarity-determining regions in a human antibody with those from a mouse.” Nature 321(6069): 522-5.
Lefranc M.-P., Pommié C. et al. (2003) “IMGT unique numbering for immunoglobulin and T-cell receptor variable domains and Ig superfamily V-like domains.” Dev. Comp. Immunol. 27, 55-77.
Leung S. O., Goldenberg D. M. et al. (1995) “Construction and characterization of a humanized, internalizing, B-cell (CD22)-specific, leukemia/lymphoma antibody, LL2.” Mol. Immunol. 32(17-18): 1413-27.
Lewis A. P. and Crowe J. S. (1991) “Immunoglobulin complementarity-determining region grafting by recombinant polymerase chain reaction to generate humanized monoclonal antibodies.” Gene 101(2): 297-302.
Lonberg N. and Huszar D. (1995) “Human Antibodies from Transgenic Mice.” Internal Review of Immunology 13: 65-93.
Morrison S. L., Johnson M. J. et al. (1984) “Chimeric human antibody molecules mouse antigen-binding domains with human constant region domains.” Proc. Natl. Acad. Sci. USA 81: 6851-55.
Neuberger M. S., Williams G. T. et al. (1985) “Recombinant antibodies possessing novel effector functions.” Nature 312(5995): 604-8.
Presta L. G., Lahr S. J. et al. (1993) “Humanization of an antibody directed against IgE.” J. Immunol. 151: 2623.
Queen C., Schneider W. P. et al. (1989) “A humanized antibody that binds to the interleukin 2 receptor.” Proc. Natl. Acad. Sci. USA 86(24): 10029-33.
Riechmann L., Clark M. et al. (1988) “Reshaping human antibodies for therapy.” Nature 332(6162): 323-7.
Rudling M., Collins V. et al. (1983) “Delivery of aclacinomycin A to human glioma cells in vitro by the low-density lipoprotein pathway.” Cancer Res. 43(10): 4600-4605.
Rudling M., Reihner E. et al. (1990) “Low Density Lipoprotein Receptor-Binding Activity in Human Tissues: Quantitative Importance of Hepatic Receptors and Evidence for Regulation of Their Expression in vivo.” PNAS 87(9): 3469-3473.
Sato K., Tsuchiya M. et al. (1994) “Humanization of a mouse anti-human interleukin-6 receptor antibody comparing two methods for selecting human framework regions.” Mol. Immunol. 31(5): 371-81.
Sims M. J., Hassal D. G. et al. (1993) “A humanized CD18 antibody can block function without cell destruction.” J. Immunol. 151: 2296.
Singer I., Kawka D. et al. (1993) “Optimal humanization of 1B4, an anti-CD18 murine monoclonal antibody, is achieved by correct choice of human V-region framework sequences.” J. Immunol. 150(7): 2844-2857.
Tokui T., Kuroiwa C. et al. (1995) “Plasma lipoproteins as targeting carriers to tumour tissues after administration of a lipophilic agent to mice.” Biopharm. Drug Dispos. 16(2): 91-103.
Tokui T., Kuroiwa C. et al. (1994) “Contribution of serum lipoproteins as carriers of antitumour agent RS-1541 (palmitoyl rhizoxin) in mice.” Biopharm. Drug Dispos. 15(2): 93-107.
Tomizuka K., Shinohara T. et al. (2000) “Double trans-chromosomic mice: maintenance of two individual human chromosome fragments containing Ig heavy and κ loci and expression of fully human antibodies.” Proc. Natl. Acad. Sci. USA 97(2): 722-727.
Verhoeyen M. and Riechmann L. (1988) “Engineering of antibodies.” Bioassays 8(2): 74-8.

1. A monoclonal antibody raised against the human LDL (Low Density Lipoprotein) receptor, binding to the peptide corresponding to amino acids 195-222 (SEQ ID NO: 1) in the peptide sequence for the human LDL receptor.

2. An antibody according to claim 1, wherein at least one CDR (Complementarity-Determining Region) of each of its light chains has a peptide sequence that is at least 70% identical to a sequence selected from among the following sequences: SEQ ID NO: 2, SEQ ID NO: 3, SEQ ID NO: 4, SEQ ID NO: 18, SEQ ID NO: 19, and SEQ ID NO: 20, and in that at least one CDR of each of its heavy chains has a peptide sequence that is at least 70% identical to a sequence selected from among the following sequences: SEQ ID NO: 5, SEQ ID NO: 6, SEQ ID NO: 7, SEQ ID NO: 21, SEQ ID NO: 22, and SEQ ID NO: 23.

3. An antibody according to claim 1, wherein each CDR in each of its light chains has a peptide sequence that is at least 70% identical to the following sequences: SEQ ID NO: 2 or SEQ ID NO: 18; SEQ ID NO: 3 or SEQ ID NO: 19; or SEQ ID NO: 4 or SEQ ID NO: 20, respectively, and in that each CDR of each of its heavy chains has a peptide sequence that is at least 70% identical to the following sequences: SEQ ID NO: 5 or SEQ ID NO: 21; SEQ ID NO: 6 or SEQ ID NO: 22; or SEQ ID NO: 7 or SEQ ID NO: 23, respectively.

4. An antibody according to claim 1, wherein the variable region of each of its light chains is coded by a nucleic acid sequence that is at least 70% identical to the nucleic acid sequence SEQ ID NO: 8, and in that the variable region of each of its heavy chains is coded by a nucleic acid sequence that is at least 70% identical to the nucleic acid sequence SEQ ID NO: 9.

5. An antibody according to claim 1, wherein said variable region of each of its light chains is coded by the nucleic acid sequence SEQ ID NO: 8, and in that said variable region of each of its heavy chains is coded by the nucleic acid sequence SEQ ID NO: 9.

6. An antibody according to claim 1, characterised in that it is an F(ab′)2 fragment, an Fab′ fragment, an Fab fragment, a CDR, or any modified version of any one of these fragments or of this region.

7. An antibody according to claim 1, characterised in that it is murine.

8. An antibody according to claim 1, characterised in that it is chimeric, humanised, or human.

9. An antibody according to claim 1, characterised in that it is coupled to a toxin.

10. An antibody according to claim 1, characterised in that it allows the recruitment of immune effector cells.

11. An antibody according to claim 1, characterised in that it allows the destruction of cancer cells.

12. An antibody according to claim 1, characterised in that it is produced in the SP2/0-AG14 mouse cell line.

13. An antibody according to claim 1, characterised in that it is produced by the H12G4 hybridoma (deposited with the CNCM under No. I-3487).

14. A stable cell line producing an antibody according to claim 1.

15. A stable cell line according to claim 14, selected from among the group consisting of: SP2/0-AG14, YB2/0, IR983F, Namalwa human myeloma, PERC6, the CHO lines, particularly CHO-K-1, CHO-Lec10, CHO-Lec1, CHO-Lec13, CHO Pro-5, CHO dhfr-, Wil-2, Jurkat, Vero, Molt-4, COS-7, 293-HEK, BHK, K6H6, NS0, SP2/0-Ag14, and P3X63Ag8.653.

16. An H12G4 hybridoma deposited with the Collection Nationale de Cultures de Microorganismes [National Microorganism Culture Collection] (CNCM) under CNCM registration number I-3487.

17. A DNA fragment having the sequence SEQ ID NO: 9 coding for the variable region of the heavy chain of an antibody according to claim 1.

18. A DNA fragment having the sequence SEQ ID NO: 8 coding for the variable region of the light chain of an antibody according to claim 1.

19. An expression vector including at least one DNA fragment selected from among the fragments having the sequence SEQ ID NO: 9 and SEQ ID NO: 8.

20. A peptide corresponding to amino acids 195-222 (SEQ ID NO: 1) in the peptide sequence of the human LDL receptor.

21. A use of an antibody according to claim 1 to activate in vitro the FcγRIII receptors of immune effector cells.

22. A use of an antibody according to claim 1 in the manufacture of a drug.

23. A use of an antibody according to claim 22 in the manufacture of a drug intended for the treatment of cancer.

24. A use according to claim 23, characterised in that the treated cancers are the cancers for which the LDL receptor is overexpressed on the surface of the cancer cells.

25. A use according to claim 23, characterised in that said cancer is prostate, pancreatic, liver, breast, stomach, ovarian, colon, or lung cancer, or leukemias.

26. A use according to claim 22 in the preparation of a drug intended for the treatment of cancers, including acute myeloid leukemia, acute monocytic leukemias, myelomonocytic leukemias, chronic myeloid leukemia in blastic crisis, lymphoid leukemias, chronic lymphoid leukemias, solid tumours such as epidermoid cervical cancer, endometrial adenocarcinoma, gastric carcinoma, hepatocellular carcinoma, choriocarcinoma, and brain tumours.

27. A pharmaceutical composition including an antibody according to claim 1 and a pharmaceutically acceptable excipient and/or carrier.

28. A use of the antibody according to claim 1 in immunohistochemical analyses of cancerous, healthy, or cirrhotic tissues, in Western-Blot or ELISA analyses, or in in vivo quantification tests.

29. A use according to claim 24, characterised in that said cancer is prostate, pancreatic, liver, breast, stomach, ovarian, colon, or lung cancer, or leukemias.


SEQUENCE LISTING (23 sequences)

SEQ ID NO: 1 - protein, 28 aa, Homo sapiens
CDGGPDCKDKSDEENCAVATCRPDEFQC

SEQ ID NO: 2 - protein, 17 aa, Mus musculus
KSSQSLLYSSSQKNYLA

SEQ ID NO: 3 - protein, 7 aa, Mus musculus
WASTRES

SEQ ID NO: 4 - protein, 8 aa, Mus musculus
QQYYTYRT

SEQ ID NO: 5 - protein, 5 aa, Mus musculus
KYGMN

SEQ ID NO: 6 - protein, 17 aa, Mus musculus
WINTNTGEPTYAEEFKG

SEQ ID NO: 7 - protein, 7 aa, Mus musculus
GGYYDSY

SEQ ID NO: 8 - DNA, 336 bp, Mus musculus
gacattgtgatgtcacagtctccatcctccctagctgtgtcagttggagagaaggttactatgagctgcaagtccagtcagagccttttatatagtagtagccaaaagaactacttggcctggtaccaacagaaaccaggacagtctcctaaagtgttgatttactgggcatccactagggaatctggggtccctgatcgcttcacaggcagtggatctgggacagatttcactctcaccatcagcagtgtgaaggctgaagacctggcagtttattactgtcagcaatattatacctatcggacgttcggtggaggcaccaaactggaaatcaaa

SEQ ID NO: 9 - DNA, 348 bp, Mus musculus
cagatccagttggtgcagtctggacctgagctgaagaagcctggagagacagtcaagatctcctgcaaggcttctgggtataccttcacaaagtatggaatgaactgggtgaagcaggctccaggaaagggtttaaagtggatgggctggataaacaccaacactggagagccaacatatgctgaagagttcaagggacggtttgccttctctttggaaacctctgccagcactgcctttttgcagatcaacaacctcaaaaatgaggacacggctacatatttctgttcaagagggggttactacgattcttactggggccaagggactctggtcactgtctctgct

SEQ ID NO: 10 - protein, 112 aa, Mus musculus
DIVMSQSPSSLAVSVGEKVTMSCKSSQSLLYSSSQKNYLAWYQQKPGQSPKVLIYWASTRESGVPDRFTGSGSGTDFTLTISSVKAEDLAVYYCQQYYTYRTFGGGTKLEIK

SEQ ID NO: 11 - protein, 116 aa, Mus musculus
QIQLVQSGPELKKPGETVKISCKASGYTFTKYGMNWVKQAPGKGLKWMGWINTNTGEPTYAEEFKGRFAFSLETSASTAFLQINNLKNEDTATYFCSRGGYYDSYWGQGTLVTVSA

SEQ ID NO: 12 - DNA, 24 bp, Homo sapiens
actgccatcaatcttccacttgac

SEQ ID NO: 13 - DNA, 23 bp, Homo sapiens
ctggacagggatccagagttcca

SEQ ID NO: 14 - DNA, 28 bp, Homo sapiens
ttgttcaagaagcacacgactgaggcac

SEQ ID NO: 15 - DNA, 30 bp, Homo sapiens
tgtcactggctcagggaaatagcccttgac

SEQ ID NO: 16 - DNA, 31 bp, Homo sapiens
caccatggagttagtttgggcagcagatcca

SEQ ID NO: 17 - protein, 28 aa, Homo sapiens
SDGGPDSKDKSDEENSAVATSRPDEFQS

SEQ ID NO: 18 - protein, 12 aa, Mus musculus
QSLLYSSSQKNY

SEQ ID NO: 19 - protein, 3 aa, Mus musculus
WAS

SEQ ID NO: 20 - protein, 8 aa, Mus musculus
QQYYTYRT

SEQ ID NO: 21 - protein, 8 aa, Mus musculus
GYTFTKYG

SEQ ID NO: 22 - protein, 8 aa, Mus musculus
INTNTGEP

SEQ ID NO: 23 - protein, 9 aa, Mus musculus
SRGGYYDSY